(19) United States
(12) Patent Application Publication
(10) Pub. No.: US 2008/0281164 A1
(43) Pub. Date: Nov. 13, 2008

(54) APPARATUS AND METHOD FOR A PATIENT MONITOR

(75) Inventors: Andrea Stebor, Tampa, FL (US); Bruce Friedman, Tampa, FL (US); John W. Booth, Tampa, FL (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/747,423

(22) Filed: May 11, 2007

(51) Int. Cl. A61B 5/00

(52) U.S. Cl. 600/300

(57) ABSTRACT

A patient monitoring apparatus is disclosed herein. The patient monitoring apparatus includes a controller, and a speaker operatively connected to the controller. The speaker is configured to selectively produce a sound adapted to audibly entertain, distract and/or soothe a patient such that the patient can be conveniently and accurately monitored. A corresponding method for monitoring one or more of a patient's vital signs is also provided.

### FIELD OF THE INVENTION

This disclosure relates generally to an apparatus and method for a patient monitor that is particularly well adapted for use with children.

### BACKGROUND OF THE INVENTION

It is well known to implement patient monitoring devices for purposes such as evaluating a patient's health and diagnosing medical conditions. Some exemplary patient monitoring devices include blood pressure monitors, thermometers, pulse oximeters, electrocardiogram (ECG) devices, and electroencephalogram (EEG) devices. Conventional patient monitoring devices generally do not differentiate between adult and pediatric patients.

One problem is that children can be uncooperative and frequently move during the monitoring process. Therefore, the clinician or parent often has to entertain or otherwise occupy the pediatric patient in order to ensure they remain generally motionless. Additionally, if the child does move during the monitoring process, the resultant data can be imprecise.

### BRIEF DESCRIPTION OF THE INVENTION

The above-mentioned shortcomings, disadvantages and problems are addressed herein which will be understood by reading and understanding the following specification.

In an embodiment, a patient monitoring apparatus includes a controller, and a speaker operatively connected to the controller. The speaker is configured to selectively produce a sound adapted to audibly entertain, distract and/or soothe a patient such that the patient can be conveniently and accurately monitored.

In another embodiment, a patient monitoring apparatus includes a controller, and a display operatively connected to the controller. The display is configured to selectively produce an image adapted to visually entertain, distract and/or soothe a patient such that the patient can be conveniently and accurately monitored.

In another embodiment, a method for monitoring one or more of a patient's vital signs includes producing a sound adapted to audibly entertain, distract and/or soothe a patient, and collecting medical data related to one or more of the patient's vital signs after the patient has been entertained, distracted and/or soothed such that the patient is less likely to interfere with the medical data collection.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a patient monitoring device operatively connected to a patient in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

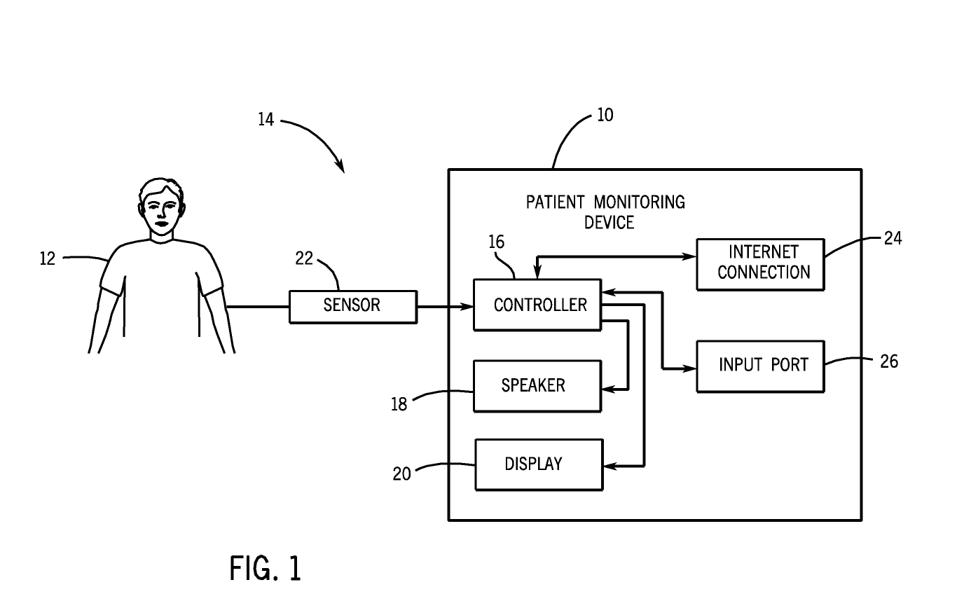

Referring to FIG. 1, a schematically represented patient monitoring device 10 is shown operatively connected to a patient 12 in accordance with an embodiment. The patient monitoring device 10 may include any device adapted to monitor, evaluate or diagnose a patient such as, for example, an electrocardiogram (ECG), an electroencephalogram (EEG) system, a blood pressure monitor, a pulse oximeter, a thermometer, etc. According to one embodiment, the patient monitoring device 10 includes any device adapted to monitor a patient's vital signs. For purposes of this disclosure, the term “vital signs” is defined to include any combination of temperature, pulse rate, blood pressure, oxygen saturation and respiratory rate. The patient monitoring device 10 is particularly well adapted for implementation with children; however, it may also be implemented with adult patients.

The patient monitoring device 10 is adapted to collect medical data from the patient 12 in a known manner. If, for example, the patient monitoring device 10 includes a non-invasive blood pressure (NIBP) monitor, the collected medical data would comprise blood pressure data that is obtainable using known auscultatory or oscillometric methods. According to one embodiment, the patient monitoring device 10 includes a patient interface 14, a controller 16, a speaker 18, and a display 20.

The patient interface 14 couples the patient 12 with the controller 16. Although the patient interface 14 is depicted in FIG. 1 as being a separate component connected to the patient monitoring device 10, it should be appreciated that the patient interface 14 may alternatively be included as an integral component of the patient monitoring device 10.

The patient interface 14 includes one or more sensors 22 that are in communication with the patient 12. The sensors 22 are configured to detect a selectable category of patient activity, and to transmit an analog signal reflective of the detected patient activity. The patient interface 14 may comprise a variety of different devices depending at least in part on the specific type of patient monitoring device 10 implemented. As one example, if the patient monitoring device 10 includes a NIBP monitor, the patient interface 14 may include a pressure transducer (not shown) operatively connected to an inflatable blood pressure cuff (not shown). According to another example, if the patient monitoring device 10 includes an ECG monitor, the patient interface 14 may include a plurality of electrodes (not shown) operatively connected to a corresponding plurality of lead wires (not shown).

According to one embodiment, the controller 16 includes a central processing unit (CPU). The controller 16 is adapted to receive input from the patient interface 14 and to process the input in a known manner. The controller 16 may, for example, be adapted to convert analog signals from the sensors 22 into digital data, to evaluate the converted digital data, and to present the converted digital data in a convenient form.

The controller 16 is also configured to selectively operate the speaker 18 and/or the display 20 in a manner adapted to entertain, distract and/or soothe the patient 12. Advantageously, by entertaining, distracting and/or soothing the patient 12, the patient 12 is more likely to remain relatively still and quiet such that the patient 12 can be accurately and conveniently monitored. As previously indicated, the patient monitoring device 10 configured in the manner described herein is particularly well adapted for use with children because they are more likely to move around during the monitoring process. Although the speaker 18 and the display 20 are shown as being an internal component of the patient monitoring device 10 in accordance with one embodiment, it should be appreciated that the speaker 18 and/or the display 20 may alternatively be externally disposed relative to the patient monitoring device 10.

The speaker 18 can produce sound adapted to audibly entertain, distract and/or soothe the patient 12. In a non-limiting manner, the sound produced by the speaker 18 may include music, animal noises, human voices, etc. The speaker 18 can be implemented in combination with the display 20 (e.g., to show a movie or television show with sound), or the speaker 18 may be implemented independently (e.g., to provide sound without an image).

The display 20 can produce images adapted to visually entertain, distract and/or soothe the patient 12. In a non-limiting manner, the images produced by the display 20 may include cartoons, pictures of animals, etc. The display 20 can be implemented in combination with the speaker 18, or the display 20 may be implemented independently (e.g., to show a series of images without sound).

The patient monitoring device 10 may optionally include an Internet connection 24 and an input port 26. The Internet connection 24 may be implemented to download additional sounds and images from the Internet. The input port 26 may include a wireless connection, and may be implemented to download additional sounds and images from an external device (not shown) including but not limited to a CD player, DVD player, other media player or other storage device. Accordingly, the Internet connection 24 and the input port 26 increase the likelihood that the patient monitoring device 10 will have access to a particular patient's optimally entertaining, distracting and/or soothing sounds and images.

According to an embodiment wherein the patient monitoring device 10 includes a NIBP monitor (not shown), the speaker 18 and/or the display 20 could initially be implemented to entertain, distract and/or soothe the patient 12. After a predetermined amount of time or after the patient becomes sufficiently stationary, an NIBP cuff (not shown) can be inflated in order to record the patient's blood pressure in a convenient manner and with minimal interference. After the blood pressure measurement has been recorded, the speaker 18 and/or the display 20 can be turned off to conserve energy and to avoid unnecessarily interrupting other nearby patients.

According to an embodiment wherein the patient monitoring device 10 includes a pulse oximeter (not shown), the speaker 18 and/or the display 20 could be implemented to entertain, distract and/or soothe the patient 12 after a pulse oximeter probe (not shown) is placed on the patient's finger and before the pulse oximeter collects any data. The speaker 18 and/or the display 20 could be turned off after the pulse oximeter data has been collected to conserve energy and to avoid unnecessarily interrupting other nearby patients.

According to an embodiment wherein the patient monitoring device 10 includes a thermometer (not shown), the speaker 18 and/or the display 20 could be implemented to entertain, distract and/or soothe the patient 12 after a temperature probe (not shown) is removed from its probe well (not shown). The speaker 18 and/or the display 20 could be turned off after the temperature data has been collected to conserve energy and to avoid unnecessarily interrupting other nearby patients.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

We claim:

1. A patient monitoring apparatus comprising:
a controller; and
a speaker operatively connected to the controller, wherein the speaker is configured to selectively produce a sound adapted to audibly entertain, distract and/or soothe a patient such that the patient can be conveniently and accurately monitored.

2. The patient monitoring apparatus of claim 1, wherein the patient monitoring device is selected from the group consisting of a blood pressure monitor, an electrocardiogram, an electroencephalogram, a thermometer, and a pulse oximeter.

3. The patient monitoring apparatus of claim 2, wherein the speaker is configured to selectively produce music adapted to audibly entertain, distract and/or soothe the patient.

4. The patient monitoring apparatus of claim 2, further comprising a display operatively connected to the controller, wherein the display is configured to selectively produce an image adapted to visually entertain, distract and/or soothe the patient such that the patient can be conveniently and accurately monitored.

5. The patient monitoring apparatus of claim 2, further comprising a patient interface coupling the controller with the patient.

6. The patient monitoring apparatus of claim 5, wherein the patient interface includes a sensor.

7. The patient monitoring apparatus of claim 2, further comprising an Internet connection coupled with the controller.

8. The patient monitoring apparatus of claim 2, further comprising an input port coupled with the controller.

9. A patient monitoring apparatus comprising:
a controller; and
a display operatively connected to the controller, wherein the display is configured to selectively produce an image adapted to visually entertain, distract and/or soothe a patient such that the patient can be conveniently and accurately monitored.

10. The patient monitoring apparatus of claim 9, wherein the patient monitoring device is selected from the group consisting of a blood pressure monitor, an electrocardiogram, an electroencephalogram, a thermometer, and a pulse oximeter.

11. The patient monitoring apparatus of claim 10, further comprising a patient interface coupling the controller with the patient.

12. The patient monitoring apparatus of claim 11, wherein the patient interface includes a sensor.

13. The patient monitoring apparatus of claim 12, further comprising a speaker operatively connected to the controller, wherein the speaker is configured to selectively produce a sound adapted to audibly entertain, distract and/or soothe the patient such that the patient can be conveniently and accurately monitored.

14. The patient monitoring apparatus of claim 13, wherein the speaker is configured to selectively produce music adapted to audibly entertain, distract and/or soothe the patient.

15. The patient monitoring apparatus of claim 14, further comprising an Internet connection coupled with the controller.

16. The patient monitoring apparatus of claim 15, further comprising an input port coupled with the controller.

17. A method for monitoring one or more of a patient's vital signs comprising:
producing a sound adapted to audibly entertain, distract and/or soothe a patient; and
collecting medical data related to one or more of the patient's vital signs after the patient has been entertained, distracted and/or soothed such that the patient is less likely to interfere with the medical data collection.

18. The method of claim 17, further comprising automatically shutting off the sound after the medical data has been collected.

19. The method of claim 17, wherein said producing a sound includes producing music.

20. The method of claim 17, further comprising producing an image adapted to visually entertain, distract and/or soothe the patient.
